(19) United States
(12) Patent Application Publication
(10) Pub. No.: US 2005/0143614 A1
(43) Pub. Date: Jun. 30, 2005

(54) Process and catalyst for C8 alkylaromatic isomerization

(76) Inventors: E. Alejandro Leon-Escamilla, Chicago, IL (US); Paula L. Bogdan, Mount Prospect, IL (US); John E. Bauer, LaGrange Park, IL (US); Gregory F. Maher, Aurora, IL (US)

Correspondence Address: JOHN G TOLOMEI, PATENT DEPARTMENT;UOP LLC, 25 EAST ALGONQUIN ROAD, P O BOX 5017, DES PLAINES, IL, 60017-5017, US

(21) Appl. No.: 10/749,156

(22) Filed: Dec. 30, 2003

(51) Int. Cl. C07C 5/22

(52) U.S. Cl. 585/481

(57) ABSTRACT

A process for isomerizing ethylbenzene into paraxylene using a zeolitic catalyst system based on MTW-type zeolite that is substantially free of mordenite. The invention obtains an improved yield of paraxylene without excess benzene production by dealkylation. The zeolitic silica-to-alumina ratio ranges from 20 to 45. Elimination of mordenite in the catalyst improves yields and integrated aromatics complex economics by reducing undesirable aromatic ring-loss reactions as well.

### FIELD OF THE INVENTION

The present invention relates to catalytic hydrocarbon conversion, and more specifically to the use of a catalyst system comprising MTW-type zeolite substantially free of mordenite in a hydrocarbon conversion process, and even more specifically to an aromatics isomerization process to convert ethylbenzene into xylene.

### BACKGROUND OF THE INVENTION

The xylenes, para-xylene, meta-xylene and ortho-xylene, are important intermediates that find wide and varied application in chemical syntheses. Para-xylene upon oxidation yields terephthalic acid that is used in the manufacture of synthetic textile fibers and resins. Meta-xylene is used in the manufacture of plasticizers, azo dyes, wood preservers, etc. Ortho-xylene is feedstock for phthalic anhydride production.

Xylene isomers from catalytic reforming or other sources generally do not match demand proportions as chemical intermediates, and further comprise ethylbenzene, which is difficult to separate or to convert. Para-xylene in particular is a major chemical intermediate with rapidly growing demand, but amounts to only 20-25% of a typical C₈ aromatics stream. Adjustment of isomer ratio to demand can be effected by combining xylene-isomer recovery, such as adsorption for para-xylene recovery, with isomerization to yield an additional quantity of the desired isomer. Isomerization converts a non-equilibrium mixture of the xylene isomers that is lean in the desired xylene isomer to a mixture approaching equilibrium concentrations.

Various catalysts and processes have been developed to effect xylene isomerization. In selecting appropriate technology, it is desirable to run the isomerization process as close to equilibrium as practical in order to maximize the para-xylene yield; however, associated with this is a greater cyclic C₈ loss due to side reactions. The approach to equilibrium that is used is an optimized compromise between high C₈ cyclic loss at high conversion (i.e., very close approach to equilibrium) and high utility costs due to the large recycle rate of unconverted C₈ aromatics. Catalysts thus are evaluated on the basis of a favorable balance of activity, selectivity and stability.

U.S. Pat. No. 4,962,258 discloses a process for liquid phase xylene isomerization over gallium-containing, crystalline silicate molecular sieves as an improvement over aluminosilicate zeolites ZSM-5, ZSM-12 (MTW-type), and ZSM-21 as shown in U.S. Pat. No. 3,856,871. The '258 patent refers to borosilicate work, as exemplified in U.S. Pat. No. 4,268,420, and to zeolites of the large pore type such as faujasite or mordenite.

U.S. Pat. No. 5,744,673 discloses an isomerization process using beta zeolite and exemplifies the use of gas-phase conditions with hydrogen. U.S. Pat. No. 5,898,090 discloses an isomerization process using crystalline silicoaluminophosphate molecular sieves.

Catalysts for isomerization of C₈ aromatics ordinarily are classified by the manner of processing ethylbenzene associated with the xylene isomers. Ethylbenzene is not easily isomerized to xylenes, but it normally is converted in the isomerization unit because separation from the xylenes by superfractionation or adsorption is very expensive. A widely used approach is to dealkylate ethylbenzene to form principally benzene while isomerizing xylenes to a near-equilibrium mixture. An alternative approach is to react the ethylbenzene to form a xylene mixture via conversion to and reconversion from naphthenes in the presence of a solid acid catalyst with a hydrogenation-dehydrogenation function. The former approach commonly results in higher ethylbenzene conversion, thus lowering the quantity of recycle to the para-xylene recovery unit and concomitant processing costs, but the latter approach enhances xylene yield by forming xylenes from ethylbenzene. A catalyst composite and process which enhance conversion according to the latter approach, i.e., achieve ethylbenzene isomerization to xylenes with high conversion, would effect significant improvements in xylene-production economics.

### SUMMARY OF THE INVENTION

A principal object of the present invention is thus to provide a process for the isomerization of alkylaromatic hydrocarbons. More specifically, the process of the present invention is directed to liquid phase isomerization for C₈ aromatic hydrocarbons over a MTW-type zeolite catalyst in order to obtain improved yields of desired xylene isomers.

The present invention is based on the discovery that a catalyst system comprising a substantially mordenite-free MTW-type zeolite with a binder demonstrates improved conversion and selectivity in C₈ aromatics isomerization, while minimizing undesired benzene formation.

Accordingly, a broad embodiment of the present invention is directed toward a process for the isomerization of alkylaromatics comprising contacting a C₈ aromatics rich hydrocarbon feed stream comprising ethylbenzene and less than the equilibrium amount of para-xylene with a catalyst comprising substantially mordenite-free MTW zeolite, preferably with a silica to alumina ratio less than about 40, at isomerization conditions to obtain a product having an increased para-xylene content relative to that of the feedstock. Preferably the MTW-containing catalyst comprises a platinum-group metal, with platinum being an especially preferred component. The optimal catalyst composite also comprises an inorganic-oxide binder, usually alumina and/or silica.

These, as well as other objects and embodiments will become evident from the following detailed description of the invention.

### DETAILED DESCRIPTION OF THE INVENTION

The feedstocks to the aromatics isomerization process of this invention comprise isomerizable alkylaromatic hydrocarbons of the general formula C₆H₍₆₋ₙ₎Rₙ, where n is an integer from 2 to 5 and R is CH₃, C₂H₅, C₃H₇, or C₄H₉, in any combination and including all the isomers thereof. Suitable alkylaromatic hydrocarbons include, for example but without so limiting the invention, ortho-xylene, meta-xylene, para-xylene, ethylbenzene, ethyltoluenes, tri-methylbenzenes, di ethylbenzenes, tri-ethyl-benzenes, methylpropylbenzenes, ethylpropylbenzenes, di-isopropylbenzenes, and mixtures thereof.

A particularly preferred application of the catalyst system of the present invention is the isomerization of a C₈ aromatic mixture containing ethylbenzene and xylenes. Generally the mixture will have an ethylbenzene content of about 1 to about 50 wt-%, an ortho-xylene content of 0 to about 35 wt-%, a meta-xylene content of about 20 to about 95 wt-% and a para-xylene content of 0 to about 30 wt-%. The aforementioned C₈ aromatics are a non-equilibrium mixture, i.e., at least one C₈ aromatic isomer is present in a concentration that differs substantially from the equilibrium concentration at isomerization conditions. Usually the non-equilibrium mixture is prepared by removal of para-, ortho- and/or meta-xylene from a fresh C₈ aromatic mixture obtained from an aromatics-production process.

The alkylaromatic hydrocarbons may be utilized in the present invention as found in appropriate fractions from various refinery petroleum streams, e.g., as individual components or as certain boiling-range fractions obtained by the selective fractionation and distillation of catalytically cracked or reformed hydrocarbons. Concentration of the isomerizable aromatic hydrocarbons is optional; the process of the present invention allows the isomerization of alkylaromatic-containing streams such as catalytic reformate with or without subsequent aromatics extraction to produce specified xylene isomers and particularly to produce para-xylene. A C₈ aromatics feed to the present process may contain nonaromatic hydrocarbons, i.e., naphthenes and paraffins, in an amount up to about 30 wt-%. Preferably the isomerizable hydrocarbons consist essentially of aromatics, to ensure pure products from downstream recovery processes. Moreover, a C₈ aromatics feed that is rich in undesired ethylbenzene can be supplied such that it can be converted to xylenes or other non-C₈ compounds in order to further concentrate desired xylene species.

According to the process of the present invention, an alkylaromatic hydrocarbon feed mixture, preferably in admixture with hydrogen, is contacted with a catalyst of the type hereinafter described in an alkylaromatic hydrocarbon isomerization zone. Contacting may be effected using the catalyst in a fixed-bed system, a moving-bed system, a fluidized-bed system, or in a batch-type operation. In view of the danger of attrition loss of the valuable catalyst and of the simpler operation, it is preferred to use a fixed-bed system. In this system, a hydrogen-rich gas and the feed mixture are preheated by suitable heating means to the desired reaction temperature and then passed into an isomerization zone containing a fixed bed of catalyst. The conversion zone may be one or more separate reactors with suitable means therebetween to ensure that the desired isomerization temperature is maintained at the entrance to each zone. The reactants may be contacted with the catalyst bed in either upward-, downward-, or radial-flow fashion, and the reactants may be in the liquid phase, a mixed liquid-vapor phase, or a vapor phase when contacted with the catalyst.

The alkylaromatic feed mixture, preferably a non-equilibrium mixture of C₈ aromatics, is contacted with the isomerization catalyst at suitable alkylaromatic-isomerization conditions. Such conditions comprise a temperature ranging from about 0° to 600° C. or more, and preferably in the range of from about 300° to 500° C. The pressure generally is from about 1 to 100 atmospheres absolute, preferably less than about 50 atmospheres. Sufficient catalyst is contained in the isomerization zone to provide a liquid hourly space velocity with respect to the hydrocarbon feed mixture of from about 0.1 to 30 h⁻¹, and preferably 0.5 to 10 hr⁻¹. The hydrocarbon feed mixture optimally is reacted in admixture with hydrogen at a hydrogen/hydrocarbon mole ratio of about 0.5:1 to about 25:1 or more. Other inert diluents such as nitrogen, argon and light hydrocarbons may be present.

The reaction proceeds via the mechanism, described hereinabove, of isomerizing xylenes while reacting ethylbenzene to form a xylene mixture via conversion to and reconversion from naphthenes. The yield of xylenes in the product thus is enhanced by forming xylenes from ethylbenzene. The loss of C₈ aromatics through the reaction thus is low: typically less than about 4 wt-% per pass of C₈ aromatics in the feed to the reactor, preferably no more than about 3.5 wt-%, and most preferably less than 3 wt-%.

The particular scheme employed to recover an isomerized product from the effluent of the reactors of the isomerization zone is not deemed to be critical to the instant invention, and any effective recovery scheme known in the art may be used. Typically, the liquid product is fractionated to remove light and/or heavy byproducts to obtain the isomerized product. Heavy byproducts include A₁₀ compounds such as dimethylethylbenzene. In some instances, certain product species such as ortho xylene or dimethylethylbenzene may be recovered from the isomerized product by selective fractionation. The product from isomerization of C₈ aromatics usually is processed to selectively recover the para-xylene isomer, optionally by crystallization. Selective adsorption is preferred using crystalline aluminosilicates according to U.S. Pat. No. 3,201,491. Improvements and alternatives within the preferred adsorption recovery process are described in U.S. Pat. No. 3,626,020, U.S. Pat. No. 3,696,107, U.S. Pat. No. 4,039,599, U.S. Pat. No. 4,184,943, U.S. Pat. No. 4,381,419 and U.S. Pat. No. 4,402,832, incorporated herein by reference.

An essential component of the catalyst of the present invention is at least one substantially mordenite-free MTW type zeolitic molecular sieve, also characterized as “low silica ZSM-12” and defined in the instant invention to include molecular sieves with a silica to alumina ratio less than about 45, preferably from about 20 to about 40. Substantially mordenite-free is herein defined to mean a component containing less than about 20 wt-% mordenite impurity, preferably less than about 10 wt-%, and most preferably less than about 5 wt-% mordenite which is about at the lower level of detect-ability using most characterization methods known to those skilled in the art such as x-ray crystallography. Applicants have surprisingly discovered that a unique and novel property of MTW-type zeolite appears when the silica to alumina ratio is lowered in comparison to the prior art, and that the avoidance of the concomitant mordenite phase under low silica conditions results in a catalysts composite with excellent properties for low aromatic ring loss when converting ethylbenzene to para-xylene under minimum benzene conditions.

The preparation of MTW-type zeolites by crystallizing a mixture comprising an alumina source, a silica source and templating agent uses methods well known in the art. U.S. Pat. No. 3,832,449, which is herein incorporated by reference, more particularly describes an MTW-type zeolite using tetraalkylammonium cations. U.S. Pat. No. 4,452,769 and U.S. Pat. No. 4,537,758, which are incorporated herein by reference, use a methyltriethylammonium cation to prepare a highly siliceous MTW-type zeolite. U.S. Pat. No. 6,652,832, which is incorporated herein by reference, use a N,N-dimethylhexamethyleneimine cation as a template to produce low silica-to-alumina ratio MTW type zeolite without MFI impurities. Preferably high purity crystals are used as seeds for subsequent batches.

The MTW-type zeolite is preferably composited with a binder for convenient formation of catalyst particles. The proportion of zeolite in the catalyst is about 1 to 90 mass-%, preferably about 2 to 20 mass-%, the remainder other than metal and other components discussed herein being the binder component.

As mentioned previously, the zeolite will usually be used in combination with a refractory inorganic oxide binder. The binder should be a porous, adsorptive support having a surface area of about 25 to about 500 m²/g. It is intended to include within the scope of the present invention binder materials which have traditionally been utilized in hydrocarbon conversion catalysts such as: (1) refractory inorganic oxides such as alumina, titania, zirconia, chromia, zinc oxide, magnesia, thoria, boria, silica-alumina, silica-magnesia, chromia-alumina, alumina-boria, silica-zirconia, phosphorus-alumina, etc.; (2) ceramics, porcelain, bauxite; (3) silica or silica gel, silicon carbide, clays and silicates including those synthetically prepared and naturally occurring, which may or may not be acid treated, for example, attapulgite clay, diatomaceous earth, fuller's earth, kaolin, kieselguhr, etc.; (4) crystalline zeolitic aluminosilicates, either naturally occurring or synthetically prepared such as FAU, MEL, MFI, MOR, MTW (IUPAC Commission on Zeolite Nomenclature), in hydrogen form or in a form which has been exchanged with metal cations, (5) spinels such as MgAl₂O₄, FeAl₂O₄, ZnAl₂O₄, CaAl₂O₄, and other like compounds having the formula MO Al₂O₃ where M is a metal having a valence of 2; and (6) combinations of materials from one or more of these groups.

A preferred refractory inorganic oxide for use in the present invention is alumina. Suitable alumina materials are the crystalline aluminas known as the gamma-, eta-, and theta-alumina, with gamma- or eta-alumina giving the best results.

A shape for the catalyst composite is an extrudate. The well-known extrusion method initially involves mixing of the molecular sieve with optionally the binder and a suitable peptizing agent to form a homogeneous dough or thick paste having the correct moisture content to allow for the formation of extrudates with acceptable integrity to withstand direct calcination. Extrudability is determined from an analysis of the moisture content of the dough, with a moisture content in the range of from about 30 to about 50 mass.% being preferred. The dough is then extruded through a die pierced with multiple holes and the spaghetti-shaped extrudate is cut to form particles in accordance with techniques well known in the art. A multitude of different extrudate shapes is possible, including, but not limited to, cylinders, cloverleaf, dumbbell and symmetrical and asymmetrical polylobates. It is also within the scope of this invention that the extrudates may be further shaped to any desired form, such as spheres, by marumerization or any other means known in the art.

An alternative shape of the composite is a sphere continuously manufactured by the well-known oil drop method. Preparation of alumina-bound spheres generally involves dropping a mixture of molecular sieve, alumina sol, and gelling agent into an oil bath maintained at elevated temperatures. Alternatively, gelation of a silica hydrosol may be effected using the oil-drop method. One method of gelling this mixture involves combining a gelling agent with the mixture and then dispersing the resultant combined mixture into an oil bath or tower which has been heated to elevated temperatures such that gelation occurs with the formation of spheroidal particles. The gelling agents that may be used in this process are hexamethylene tetraamine, urea or mixtures thereof. The gelling agents release ammonia at the elevated temperatures which sets or converts the hydrosol spheres into hydrogel spheres. The spheres are then continuously withdrawn from the oil bath and typically subjected to specific aging treatments in oil and an ammoniacal solution to further improve their physical characteristics.

Preferably the resulting composites are then washed and dried at a relatively low temperature of about 50-200° C. and subjected to a calcination procedure at a temperature of about 450-700° C. for a period of about 1 to about 20 hours.

Catalysts of the invention also comprise a platinum-group metal, including one or more of platinum, palladium, rhodium, ruthenium, osmium, and iridium. The preferred platinum-group metal is platinum. The platinum-group metal component may exist within the final catalyst composite as a compound such as an oxide, sulfide, halide, oxysulfide, etc., or as an elemental metal or in combination with one or more other ingredients of the catalyst composite. It is believed that the best results are obtained when substantially all the platinum-group metal component exists in a reduced state.

The platinum-group metal component may be incorporated into the catalyst composite in any suitable manner. One method of preparing the catalyst involves the utilization of a water-soluble, decomposable compound of a platinum-group metal to impregnate the calcined sieve/binder composite. Alternatively, a platinum-group metal compound may be added at the time of compositing the sieve component and binder. Complexes of platinum group metals which may be employed in impregnating solutions, co-extruded with the sieve and binder, or added by other known methods include chloroplatinic acid, chloropalladic acid, ammonium chloroplatinate, bromoplatinic acid, platinum trichloride, platinum tetrachloride hydrate, platinum dichlorocarbonyl dichloride, tetramine platinic chloride, dinitrodiaminoplatinum, sodium tetranitroplatinate (II), palladium chloride, palladium nitrate, palladium sulfate, diaminepalladium (II) hydroxide, tetraminepalladium (II) chloride, and the like. It is within the scope of the present invention that the catalyst composites may contain other metal components. Such metal modifiers may include rhenium, tin, germanium, lead, cobalt, nickel, indium, gallium, zinc, uranium, dysprosium, thallium, and mixtures thereof. Catalytically effective amounts of such metal modifiers may be incorporated into the catalysts by any means known in the art to effect a homogeneous or stratified distribution.

The catalysts of the present invention may contain a halogen component, comprising either fluorine, chlorine, bromine or iodine or mixtures thereof, with chlorine being preferred. Preferably, however, the catalyst contains no added halogen other than that associated with other catalyst components.

The catalyst composite is dried at a temperature of from about 100° to about 320° C. for a period of from about 2 to about 24 or more hours and, usually, calcined at a temperature of from about 400° to about 650° C. in an air atmosphere for a period of from about 0.1 to about 10 hours until the metallic compounds present are converted substantially to the oxide form. If desired, the optional halogen component may be adjusted by including a halogen or halogen-containing compound in the air atmosphere.

The resultant calcined composites optimally are subjected to a substantially water-free reduction step to ensure a uniform and finely divided dispersion of the optional metallic components. The reduction optionally may be effected in the process equipment of the present invention. Substantially pure and dry hydrogen (i.e., less than 20 vol. ppm H2O) preferably is used as the reducing agent in this step. The reducing agent contacts the catalyst at conditions, including a temperature of from about 200° to about 650° C. and for a period of from about 0.5 to about 10 hours, effective to reduce substantially all of the Group VIII metal component to the metallic state. In some cases the resulting reduced catalyst composite may also be beneficially subjected to presulfiding by a method known in the art such as with neat H₂S at room temperature to incorporate in the catalyst composite from about 0.05 to about 1.0 wt-% sulfur calculated on an elemental basis.

### EXAMPLES

The following examples are presented only to illustrate certain specific embodiments of the invention, and should not be construed to limit the scope of the invention as set forth in the claims. There are many possible other variations, as those of ordinary skill in the art will recognize, within the spirit of the invention.

### Example I

Samples of catalysts comprising zeolites were prepared for comparative pilot-plant testing. First, a catalyst A was prepared to represent a prior art catalyst for use in a process of isomerization of ethylbenzene to para-xylene with minimal benzene formation.

Catalyst A contained SM-3 silicoaluminophosphate prepared according to the teachings of U.S. Pat. No. 4,943,424 and had characteristics as disclosed in the '424 patent. Following the teachings of U.S. Pat. No. 5,898,090, catalyst A was composited with alumina and tetramine platinic chloride at a platinum levels of 0.28 wt-% on an elemental basis. The composite comprised about 60 wt-% SM-3 and 40 wt-% alumina, and then the catalyst was calcined and reduced, with the product labeled as Catalyst A.

### Example II

Catalysts of the invention were prepared containing MTW-type zeolite prepared in accordance with U.S. Pat. No. 4,452,769, but achieving varying amounts of mordenite impurity. To a solution of 0.4 grams sodium hydroxide in 9 grams distilled water was added 0.078 g aluminum hydroxide hydrate and stirred until dissolved. A second solution of 1.96 grams of methyltriethylammonium halide (MTEA-Cl, note here the chloride form was used instead of the bromide form) in 9 grams distilled water was prepared and stirred until dissolved. Then, both solutions were stirred together until homogenized. Next, 3 grams of precipitated silica was added, stirred for 1 hour at room temperature and sealed in a Teflon-lined autoclave for 8 days at 150° C. Zeolite type MTW was recovered after cooling, filtering, and washing with distilled water. After drying a product of 5 Na₂O:1.25Al₂O₃:50SiO₂: 1000H₂O: 10(MTEA-Cl) with a BET 454 m²/g, was obtained. X-ray diffraction analysis indicated that the product was 100 wt-% MTW type zeolite.

To form catalyst B, about 10 wt % of the dry 100 wt-% MTW-zeolite was composited with about 90 wt % alumina to form extruded shaped catalyst particles. The particles were then metal-impregnated using a solution of tetraamine platinum chloride. Upon completion of the impregnation, the catalyst was dried, oxidized, reduced, and sulfided to yield a catalyst containing about 0.3 wt-% platinum and 0.1 wt-% sulfur. The finished catalyst was labeled catalyst B.

### Example III

Catalysts A and B were evaluated for ethylbenzene isomerization to para-xylene using a pilot plant flow reactor processing a non-equilibrium C₈ aromatic feed having the following approximate composition in wt-%:

| | |
|---|---|
| Toluene | 0.2 |
| C₈ Non-aromatics | 8.3 |
| Ethylbenzene | 26.8 |
| Para-xylene | 0.9 |
| Meta-xylene | 42.4 |
| Ortho-xylene | 21.0 |
| C₉⁺ Non-aromatics | 0.4 |

This feed was contacted with catalyst at a pressure of about 620 kPa, a liquid hourly space velocity of 4, and a hydrogen/hydrocarbon mole ratio of 4. Reactor temperature was adjusted to effect a favorable conversion level. Conversion is expressed as the disappearance per pass of ethylbenzene, and C₈ aromatic ring loss is primarily to benzene and toluene, with smaller amounts of light gases being produced. Results were as follows:

| Catalyst | A | B |
|---|---|---|
| Temperature ° C. | 375 | 371 |
| p-xylene/xylenes | 22.3 | 22.3 |
| EB conversion, wt-% | 30 | 35 |
| Benzene yield, wt-% | 1.4 | 0.6 |
| C₈ Ring loss | 2.5 | 2.5 |

Accordingly, catalyst B showed better conversion of ethylbenzene while minimizing the yield of undesired benzene as compared to catalyst A of the prior art. Note that the “C₈ ring loss” is in mol % defined as “(1-(C₈ naphthenes and aromatics in product)/(C₈ naphthenes and aromatics in feed))*100”, which represents material that has to be circulated to another unit in an aromatics complex. Such circulation is expensive and a low amount of C8 ring loss is a favorable feature of the catalyst of the present invention.

### Example IV

Similarly, additional batches of MTW-type zeolite were prepared according the procedure outlined above in Example II. However due to variations in stirring and seed crystals as well as other inhomogeneous effects among the vessels used, resulting batches were discovered to have various amounts of impurities at a silica-to-alumina ratio of about 34. The impurities were determined to be a mordenite-type zeolite by using x-ray diffraction methods. To understand the effect of the impurity, various samples were obtained and made into catalysts.

Catalyst C was prepared with the same material as Catalyst B, 100 wt-% MTW. Catalyst D was prepared with a zeolitic composite comprising 90 wt-% MTW and 10 wt-% mordenite. Catalyst E was prepared with a zeolitic composite comprising 80 wt-% MTW and 20 wt-% mordenite. Finally, Catalyst F was prepared with a zeolitic composite comprising 50 wt-% MTW and 50 wt-% mordenite to illustrate a catalyst with substantial mordenite impurity and thus is not considered a catalyst within the scope of the invention.

Catalysts C through F were formed into extruded particles using about 5 wt-% of the zeolitic composite material above and about 95 wt-% alumina binder. The particles were then metal-impregnated using a solution of tetraamine platinum chloride. Upon completion of the impregnation, the catalysts was dried, oxidized, reduced, and sulfided to yield catalysts containing about 0.3 wt-% platinum and 0.1 wt-% sulfur. The finished catalysts were labeled respectively, catalysts C through F.

### Example V

Catalysts C through F were evaluated for C₈ aromatic ring loss using a pilot plant flow reactor processing a non-equilibrium C₈ aromatic feed having the following approximate composition in wt-%:

| | |
|---|---|
| C8 Non-aromatics | 7 |
| Ethylbenzene | 16 |
| Para-xylene | <1 |
| Meta-xylene | 52 |
| Ortho-xylene | 25 |

This feed was contacted with of catalyst at a pressure of about 620 kPa, a liquid hourly space velocity of 4, and a hydrogen/hydrocarbon mole ratio of 4. Reactor temperature was adjusted between about 370 to 375° C. to effect a favorable ethylbenzene conversion level. Results were as follows:

Catalyst

| | C | D | E | F |
|---|---|---|---|---|
| p-xylene/xylenes | 22.3 | 22.3 | 22.3 | 22.3 |
| C₈ Ring loss | 2.6 | 3.3 | 3.6 | 5.4 |

Accordingly, catalyst C showed minimum ring loss, and catalysts D thru F illustrated that mol-% ring loss increased with mordenite impurity level. Such circulation is expensive and a low amount of C8 ring loss is a favorable feature of the catalysts of the present invention, which is substantially free of the mordenite impurity.

1. A process for the isomerization of a feed mixture of xylenes and ethylbenzene comprising contacting the feed mixture in the presence of hydrogen in an isomerization zone with a catalyst comprising from about 0.1 to 2 wt-% of a platinum-group component calculated on an elemental basis, from about 1 to 90 wt-% of a substantially mordenite-free MTW-type zeolite component, having a silica-to-alumina mole ratio of about 45 or less, and an inorganic-oxide binder component at isomerization conditions comprising a temperature of from about 300° to 500° C., a pressure of from about 1 to 50 atmospheres, a liquid hourly space velocity of from about 0.5 to 10 hr⁻¹ and a hydrogen-to-hydrocarbon mole ratio of from about 0:5:1 to 25:1 to obtain an isomerized product comprising a higher proportion of para-xylene than in the feed mixture with a C₈ aromatics ring loss relative to the feed mixture of less than about 4 mol-%.

2. The process of claim 1 wherein the zeolite silica to alumina ratio is in the range from about 20 to about 40.

3. The process of claim 1 wherein the substantially mordenite-free MTW-type zeolite component comprises less than about 20 wt-% mordenite.

4. The process of claim 3 wherein the substantially mordenite-free MTW-type zeolite component comprises less than about 10 wt-% mordenite.

5. The process of claim 4 wherein the substantially mordenite-free MTW-type zeolite component comprises less than about 5 wt-% mordenite

6. The process of claim 1 wherein ortho-xylene is recovered from one or both of the isomerized product and the feed mixture.

7. The process of claim 1 further comprising recovery of para-xylene by selective adsorption from the isomerized product.

8. The process of claim 1 wherein the platinum-group metal is platinum.

9. The process of claim 1 wherein the inorganic-oxide binder component comprises one or both of alumina and silica.

10. The process of claim 9 wherein the binder is alumina.

11. The process of claim 1 wherein the MTW-type zeolite component is present in the catalyst in an amount from about 2 wt-% to about 20 wt-%.

12. The process of claim 1 wherein the isomerized product yields benzene in an amount of less than about 1 wt-% of the feed mixture.

13. The process of claim 1 wherein the C₈ aromatics ring loss relative to the feed mixture is less than about 3.5 mol-%.

14. The process of claim 13 wherein the C₈ aromatics ring loss relative to the feed mixture is less than about 3 wt-%.

15. A process for the isomerization of a feed mixture of xylenes and ethylbenzene comprising contacting the feed mixture in the presence of hydrogen in an isomerization zone with a catalyst comprising from about 0.1 to 2 wt-% of a platinum-group component calculated on an elemental basis, from about 2 to 20 wt-% of a substantially mordenite-free MTW-type zeolite component, having a silica-to-alumina mole ratio of about 20 to 45, and an inorganic-oxide binder component at isomerization conditions comprising a temperature of from about 300° to 500° C., a pressure of from about 1 to 50 atmospheres, a liquid hourly space velocity of from about 0.5 to 10 hr⁻¹ and a hydrogen-to-hydrocarbon mole ratio of from about 0:5:1 to 25:1 to obtain an isomerized product comprising a higher proportion of para-xylene than in the feed mixture with a C₈ aromatics ring loss relative to the feed mixture of less than about 3.5 mol-%.

16. The process of claim 15 wherein the substantially mordenite-free MTW-type zeolite component comprises less than about 10 wt-% mordenite.

17. The process of claim 15 wherein the isomerized product yields benzene in an amount of less than about 1 wt-% of the feed mixture.

18. A catalyst for isomerization of ethylbenzene into para-xylene with minimum benzene production, said catalyst comprising from about 0.1 to 2 wt-% of a platinum-group component calculated on an elemental basis, from about 1 to 90 wt-% of a substantially mordenite-free MTW-type zeolite component, having a silica-to-alumina mole ratio of about 45 or less, and an inorganic-oxide binder.

19. The catalyst of claim 18 further comprising from about 0.05 to about 1.0 wt-% sulfur.

20. The catalyst of claim 18 wherein the substantially mordenite-free MTW-type zeolite component comprises less than about 10 wt-% mordenite.
